(19) United States
(12) Patent Application Publication
(10) Pub. No.: US 2016/0250063 A1
(43) Pub. Date: Sep. 1, 2016

(54) Novel materials

(71) Applicant: ONBONE OY, Helsinki (FI)

(72) Inventor: Antti Pärssinen, Helsinki (FI)

(21) Appl. No.: 15/030,869

(22) PCT Filed: Oct. 21, 2014
(86) PCT No.: PCT/FI2014/050792

§ 371 Date: Apr. 21, 2016

(30) Foreign Application Priority Data
Oct. 21, 2013 (FI) 20136038

(51) Int. Cl. A61F 5/058; A61L 15/12

(52) U.S. Cl. CPC A61F 5/058; A61L 15/12; A61F 5/05841

(57) ABSTRACT

Novel composite materials, methods of producing the same and uses thereof. The composite materials which have the shape of three-dimensional object comprise a first component formed by a thermoplastic polymer selected from the group of biodegradable polyesters and mixtures thereof, and a second component formed by particles of a woody material, having a smallest dimension greater than 0.1 mm which reinforce the polyester. The material also has regions of elasticity or softness to provide for objects having properties of flexibility or semi-rigidity in at least one dimension. The material semi-rigid materials can be molded and worked at temperatures below 70° C. and are suitable for splints and circumferential casts.

### TECHNICAL FIELD

The present invention relates to wood-plastic composite materials. In particular, the present invention concerns composite materials comprising a thermoplastic polymer and a reinforcing component, which composite materials exhibit mechanical properties in the range from flexible to semi-rigid. Methods of producing such materials as well as uses of the materials are also disclosed.

### BACKGROUND ART

Casting is the most common form of external splinting and it is used for a wide array of bone and soft-tissue injuries. In this context, the function of the cast is to immobilize and to protect the injury and, especially, to minimize motion across a fracture site.

A number of casting materials are known. The first generation of casting material is formed by plaster-of-Paris (in the following abbreviated “POP”). Largely owing to its low cost and ease of molding it has gained universal acceptance. There are, however, a number of disadvantages of POP, including long setting times, messy application, low strength and relative heaviness. Although setting takes only a few minutes, drying may take many hours or days, especially if the atmosphere is moist and cool. Impacts on the plaster while it is setting may cause a weakening of the material. Furthermore, the transparency to X-rays (in the following “radiolucency”) is poor.

The second generation of casting materials is formed by synthetic composite materials, such as fiberglass reinforced polyurethane resins. They are useful alternatives to conventional plaster-of-Paris and are gaining increasing popularity. Fiberglass and resinous materials can safely be applied as external splints. These materials are lightweight, durable and waterproof but require protective packaging and some indications are difficult to apply. Further on, some of the fiberglass casting materials during applying requires special gloves for avoiding penetration of small fiberglass particles through skin. In addition, synthetic casting materials may have a shorter setting and solidification time than traditional plaster-based materials. Further, they are much more expensive than plaster at present, but to balance this disadvantage, fewer bandages are required and they are much more durable in everyday use. They are also more radiolucent than plaster based casting materials.

Further developments in the field of casting techniques have included the idea of the application of semi-rigid and rigid materials to a cast (focused rigidity), as that enables controlled functional load and stress structures in casts which thus improved restoration of the function of the affected limb. As an evidence of this casting technique Softcast (3M product) is widely used material in fracture treatment. It remains semi-rigid and flexible for resilient support in the treatment of soft tissue injuries and orthopaedic casting. Even though offering variable support and patient mobility the products of this group possess the typical disadvantages to chemically hardening thermoset materials: messy application, hazardous polymer matrix, irreversible setting and sharp edges of a cut splint.

In addition to the above mentioned synthetic toxic casting materials, various composites consisting of polycaprolactone homopolymers (PCLs) and fibrous materials are known. Examples of such materials can be found in WO 2006/027763A2, WO 2007/035875, US 2008/0262400, US 2012/0071590 and WO 2012032226. Some of the materials have orthopedic applications.

WO 94/03211 discloses a composite of cellulosic filler and polycaprolactone having improved moisture-permeability. WO2006/027763A2 discloses geometrically apertured splint manufactured from PCL and a lignocellulose filler. WO 2007/035875 discloses a cross-linked thermoplastic material with aramide fibers, into which some wood pulp or natural fibers has been incorporated. US Patent Application No. 2008/0103423 concerns a combination of cork and polycaprolactone which exhibits some degree of flexibility. Published Patent Application US 2012/0071590 describes composite material comprising hard wood chips and high molecular weight polycaprolactone useful for fracture management of upper and lower limbs. WO 2012/032226 discloses bandaging materials with textile layers.

### SUMMARY OF INVENTION

### Technical Problem

For some orthopedic immobilization applications it is required that a body support or parts of it are flexible or elastic. This is the case especially in large splints and in circumferential casts in order to increase the patient comfort. The known PCL based thermoplastic composite materials are rigid, and do not allow freedom of movements and swelling of the limbs in orthopedic applications.

There is a need for materials which, while exhibiting the advantageous properties of thermoplastic/wood particle based composites, also have sufficient flexibility for use in semi-rigid immobilization of treated bony premises.

It is an aim of the present invention to provide novel composite materials in the shape of three-dimensional objects exhibiting at least in one dimension mechanical properties extending from flexible to semi-rigid.

It is another aim of the present invention to provide methods of producing such materials.

It is still a further aim of the present invention to provide for the use of the novel flexible or semi-rigid materials.

### Solution to Problem

The present invention is based on the concept of providing composite materials by combining a first component formed by a rigid thermoplastic polymer, a second component formed by a reinforcing material and introducing into the composite materials regions of elasticity or softness to provide for objects having properties of flexibility or semi-rigidity in at least one dimension.

The composition is moldable and workable at temperatures below 70° C.

Compositions of the indicated kind can be produced by incorporating into the composite materials a third component formed by an elastic or soft thermoplastic polymer. Such a polymer can be homogeneously distributed within the polymer of the first component, or the polymer can be used to provide regions rich in said third component within the composition. In addition to, or instead of, a third component, perforations can be provided for example in the form of incisions, in particular unidirectional incisions to achieve the properties of increased flexibility or semi-rigidity.

The novel materials can be shaped into blanks or bandages or other three-dimensional products or objects which can be used for orthopedic immobilization wherein the body support or parts of it are flexible or elastic.

Examples of suitable application include splints and circumferential casts.

More specifically, the present composite products are characterized by what is stated in the characterizing part of claim 1.

The method according to the present invention is characterized by what is stated in the characterizing part of claim 31.

The novel use according to the present invention is characterized by what is stated in the characterizing part of claim 39.

### Advantageous Effects of Invention

The materials of the present invention, which are moldable and workable at temperatures below 70° C., can be heated to working temperature at which the composition can be easily formed by hand to various 3D shapes for example to contour the human anatomy. The composition solidifies upon cooling. A semi-rigid, typically at least partially flexible or elastic structure is obtained which can be readily achieved by replacing partly the hard polymer component by a polymer which is miscible with the flexible or elastic or soft polymer to bring the desired semi-rigidy and flexibility in a set product

The materials can be used for therapy and for sports applications. They allow small movements of an immobilized limb or body part. User or patient comfort is greatly increased compared to conventional rigid splints or circumferential casts.

These materials can also be combined easily with the similar rigid compositions allowing the clinician to choose the level of rigidity necessary to injury site.

When the material shaped into a planar blank it typically exhibits polymer rich longitudinal regions for improving properties of anatomical shape ability (contouring) and optionally reducing possible shape memory of the blank

Optionally the composition according to invention can be re-heated an unlimited number of times after the form has settled. In other words, formability is reversible without the composition being damaged.

In embodiments where the material exhibits incisions, breathability is further improved.

Next, embodiments will be examined with reference to the attached drawings.

### BRIEF DESCRIPTION OF DRAWINGS

FIG. 1 is a schematic presentation of one possible incision pattern applied to a rectangular blank formed by the present material;

FIG. 2 shows photographs of incised WOODCAST® test specimens, stretched at various stretching ratios;

FIG. 3 shows the effect of stretching on the pore area of incised composite materials;

FIG. 4 is a schematic presentation of the opening process of incision.

FIG. 5 illustrates the cutting patterns when preparing Ring Stiffness specimens for Examples 1 and 2 (Table 1).

### DESCRIPTION OF EMBODIMENTS

In the present context, the term “moldability” means that a composition can be heated to working temperatures at which the composition can be easily formed by hand to various 3D shapes, for example to contour the human anatomy, and the composition solidifies to a semi-rigid structure upon cooling.

“Soft” when used in the context of a polymer means that the polymer, either a thermoplastic or thermosetting polymer, has Shore D hardness 27 or less at ambient temperature.

“Hard” is a polymer which has Shore D hardness greater than 27.

“Rigid” when used in the context of a polymer means that the polymer is essentially non-flexible under conventional forces exerted by a person's limb or body part immobilized by the material for example in the shape of a splints or circumferential casts. “Semi-rigid” when used in the context of the present composite materials means that the material, under the same conditions and forces, allows from some movement in at least one direction.

“Ambient temperature” stands for a temperature of about 10 to 30° C., in particular about 15 to 25° C.

“Region” when used in connection of elasticity or softness of the composite material denotes a portion of the material. The region may extend only to a limited depth of the material or it may extend through the material in at least one dimension. The region may comprise an elongated, essentially integral area. The region may also comprise one or several isolated portions of, for example, material different from the material surrounding the isolated portion(s). “Region” may also be a portion evenly distributed throughout the composite material. Thus, a soft or elastic polymer can be homogeneous blended or mixed with the polymer of the first component to extend the region of elasticity or softness to cover essentially the whole superficial area of an object formed by the composite material.

A property of “elasticity” or “softness” can be measured by a ring stiffness test, and such a property will be manifested in a greatly reduced stiffness. Typically the stiffness will be at least 20% lower, preferably at least 30% lower than for a corresponding material, wherein the same (±10%) volume as taken up by the soft or flexible polymer of the present materials is formed by the hard polymer, for example and typically by the thermoplastic polyester or other polymer having melting point or softening point below 70° C. and higher or equal to about 55° C.

“Biodegradable” typically stands for materials which will decompose to 70% during 28 days at ambient temperature.

As discussed above, a novel composite material according to the present technology comprises generally a material which is shaped into a three-dimensional object.

Examples of such objects include planar blanks having two opposite at least essentially planar surfaces. The blanks have a length of typically 10 to 2500 mm, a width or breadth of typically 5 to 1000 mm and a thickness of typically 0.1 to 100 mm. The blank can also be a bandage or tape which for example has a length of 0.5-10 m, thickness of 0.5-1.5 mm and width of 2.5-15 cm.

In addition to planar structures, also other three-dimensional structures are possible, such as cylindrically and conically or even spherical objects as well as various chute-shaped objects, each of these optionally having one or more bent portions.

As will be discussed in more detail below, in particularly preferred embodiments, wherein the present composite material is shaped into a generally elongated, planar object which subsequently can be formed, e.g. into a tubular shape or as a chute, the planar object exhibits increased flexibility or softness in transversal direction, i.e. perpendicular to the longitudinal axis of the plane.

The present composite material comprises a first component formed by a polymer and a second component formed by a reinforcing material. The first component comprises typically a thermoplastic polymer selected from the group of biodegradable polyesters and mixtures thereof. The second component comprises particles of a woody material, having a smallest dimension greater than 0.1 mm.

According to an aspect of the present invention, the first component forms the matrix of the composite, whereas the microstructure of the second component in the composition is discontinuous. The particles of the second component can have random orientation or they can be arranged in a desired orientation. The desired orientation may be a predetermined orientation.

According to a preferred embodiment, a polycaprolactone polymer (in the following also abbreviated “PCL”) is used as a thermoplastic polymer in the first component of the composition. The polycaprolactone polymer is formed by repeating units derived from epsilon caprolactone monomers. The polymer may be a copolymer containing repeating units derived from other monomers, such as lactic acid, glycolic acid, but preferably the polymer contains at least 80% by volume of epsilon caprolactone monomers, in particular at least 90% by volume and in particular about 95 to 100% epsilon caprolactone monomers.

In a preferred embodiment, the thermoplastic polymer is selected from the group of epsilon-caprolactone homopolymers, blends of epsilon-caprolactone homopolymers and other biodegradable thermoplastic homopolymers, with 5-99 wt %, in particular 40 to 99wt %, of an epsilon-caprolactone homopolymer and 1-95 wt %, in particular 1 to 60 wt %, of a biodegradable thermoplastic polymer, and copolymers or block-copolymers of epsilon-caprolactone homopolymer and any thermoplastic biodegradable polymer, with 5 to 99wt %, in particular 40 to 99 wt % of repeating units derived from epsilon-caprolactone and 1 to 95 wt-%, in particular 1 to 60 wt-%, repeating units derived from other polymerizable material.

Examples of other biodegradable thermoplastic polymers include polylactides, poly(lactic acid), polyglycolides as well as copolymers of lactic acid and glycolic acid.

The first polymer component, in particular the epsilon caprolactone homo- or copolymer, has an average molecular weight of 60,000 to 500,000 g/mol, for example 65,000 to 300,000 /mol, in particular at least 80,000 g/mol, preferably higher than 80,000 and up to 250,000.

The molding properties of the present invention can be determined by the average molecular weight (Mn) of the polymer, such as epsilon caprolactone homo- or copolymer. A particularly preferred molecular weight range for the Mn value of PCL is from about 100,000 to about 200,000 g/mol. The number average molar mass (Mn) and the weight average molar mass (Mw) as well as the polydispersity (PDI) were measured by gel permeation chromatography. Samples for GPC measurements were taken directly from the polymerization reactor and dissolved in tetrahydrofuran (THF). The GPC was equipped with a Waters column set styragel HR(1,2 and 4) and a Waters 2410 Refractive Index Detector. THF was used as eluent with a flow rate of 0.80 ml/min at a column temperature of 35° C. A conventional polystyrene calibration was used. In determination of the water content of the monomer at different temperatures a Metroohm 756 KF Coulo meter was used.

The properties of moldability of the present composition can also be determined by the viscosity value of the polymer. For an epsilon caprolactone homopolymer: when the inherent viscosity (IV)-value of PCL is less than 1 dl/g the composite is sticky, flows while formed and forms undesired wrinkles while cooling. When PCL having IV-value closer to 2 dl/g is used the composite maintains its geometry during molding on the patient and it may be handled without adhesive properties. Thus, IV values in excess of 1 dl/g are preferred, values in excess to 1.2 dl/g are preferred and values in excess of 1.3 dl/g are particularly suitable. Advantageously the values are in the range of about 1.5 to 2.5 dl/g, for example 1.6 to 2.1 dl/g. Inherent Viscosity values were determined by LAUDA PVS 2.55 d rheometer at 25° C. The samples were prepared by solvating 1 mg of PCL in 1 ml chloroform (CH₃Cl).

A particularly important feature of the thermoplastic polymer is the viscosity which is relatively high, typically at least 1,800 Pas at 70° C., 1/10 s; the present examples show that the viscosity can be on the order of 8,000 to 13,000 Pas at 70° C., 1/10 s (dynamic viscosity, measured from melt phase). Below the indicated value, a reinforced material readily wrinkles during forming it on a patient.

The thermoplastic material is preferably a biodegradable polymer (only) but also non-biodegradable polymers may be utilized. Examples of such polymers include polyolefins, e.g. polyethylene, polypropylene, and polyesters, e.g. poly(ethylene terephthalate) and poly(butylenes terephthalate) and polyamides. The polymer may also be any cross-linked polymers manufactured prior to processing or in situ during the compounding process for example by means of ionizing radiation or chemical free-radical generators. Examples of such polymers are cross-linked polyesters, such as polycaprolactone.

Combinations of the above biodegradable polymers and said non-biodegradable polymers can also be used. Generally, the weight ratio of biodegradable polymer to any non-biodegradable polymer is 100:1 to 1:100, preferably 50:50 to 100:1 and in particular 75:25 to 100:1. Preferably, the composite material has biodegradable properties greater, the material biodegrades quicker or more completely, than the thermoplastic material alone.

By using an additional polymer component in the polymer material of the first component, mechanical properties of the first component can be improved. Such mechanical properties include tear-resistance.

According to the invention, a polymer of the afore-said kind is preferably moldable at a temperature as low as +58° C., in particular at +65° C. or slightly above, and it can be mixed with wood particles or generally any porous material gaining increased rigidity of the formed composite. The polymer component, such as polycaprolactone homopolymer, defines the form of the splinting material against the skin.

In one embodiment, the first polymer component has a melt flow index of about 0.3-2.3 g/min (at 80° C.; 2.16 kg).

The composite material according to the present technology typically exhibits formability at a temperature of about 50 to 70° C. and it is rigid at a temperature of less than 50° C., in particular at ambient temperature up to at least 45° C.

The second component is a reinforcing material which comprises or consists essentially of a woody material having a smallest diameter of greater than 0.1 mm. There can also be other wood particles present in the second component. The woody material can be granular or platy. According to one embodiment, the second component comprises a woody material derived from platy wood particles having a smallest diameter of greater than 0.1 mm. Thus, generally, the wood component can be characterized generally as being greater in size than powder.

The size and the shape of the wood particles may be regular or irregular. Typically, the particles have an average size (of the smallest dimension) in excess of 0.1 mm, advantageously in excess of 0.4 mm, for example in excess of 0.5 mm, suitably about 0.6 to 10 mm. The length of the particles (longest dimension of the particles) can vary from a value of greater than 0.6 mm to value of about 1.8 to 200 mm, for example 3 to 21 mm. The woody particles can be granular, platy or a mixture of both. Woody particles considered to be granular have a cubic shape whose ratio of general dimensions are on the order of thickness:width:length=1:1:1. In practice it is difficult to measure each individual particle to determine if it is a perfect cube. Therefore, in practice, particles considered to be granular are those where one dimension is not substantially different from the other two.

Woody particles considered to be platy means that they have generally a plate-shaped character, although particles of other forms are often included in the material. The ratio of the thickness of the plate to the smaller of the width or length of the plate's edges is generally 1:1 to 1:500, in particular about 1:2 to 1:50. Preferably, the woody particles include at least 10% by weight of chip-like particles, in which the ratio of general dimension are on the order of thickness:width:length=1:1-20:1-100, with at least one of the dimension being substantially different than another.

Based on the above, the platy particles of the present invention generally comprise wood particles having at least two dimensions greater than 1 mm and one greater than 0.1 mm, the average volume of the wood particles being generally at least 0.1 mm3 more specifically at least 1 mm³.

“Derived from platy wood particles” designates that the wood particles may have undergone some modification during the processing of the composition. For example, if blending of the first and second components is carried out with a mechanical melt-mixing device or with extruder having small nozzle dimensions, some of the original platy wood particles may be deformed to an extent.

The majority of wood particles greater in size than powder, which particles may be granular or platy, typically make up more than 70% of the woody material. The wood species can be freely selected from deciduous and coniferous wood species alike: beech, birch, alder, aspen, poplar, oak, cedar, Eucalyptus, mixed tropical hardwood, pine, spruce and larch tree for example. Other suitable raw-materials can be used, and the woody material of the composite can also be any manufactured wood product.

The particles can be derived from wood raw-material typically by cutting or chipping of the raw-material. Wood chips of deciduous or coniferous wood species are preferred, such as chips of aspen or birch.

In addition to wood chips and other platy particles, the present composition can contain reinforcing fibrous material, for example cellulose fibers, such as flax or seed fibers of cotton, wood skin, leaf or bark fibers of jute, hemp, soybean, banana or coconut, stalk fibers (straws) of hey, rice, barley and other crops and plants including plants having hollow stem which belong to main class of Tracheobionta and e.g. the subclass of meadow grasses (bamboo, reed, scouring rush, wild angelica and grass).

The composition may contain particulate or powdered material, such as sawdust, typically having particles with a size of less than 0.5 mm*0.5 mm*0.5 mm. Particulate or powdered material is characterised typically as material of a size in which the naked eye can no longer distinguish unique sides of the particle. Platy particles are easily recognizable as one dimension is recognizable by the naked eye as being larger than another. Granular particles, while having substantially equal dimensions, are of such dimension that their unique sides can be determined by the naked eye and oriented.

In one embodiment, the woody material comprises platy wood particles or particles obtained from such wood particles, by crushing, said particles forming about 30 to 100% of the total weight of the second component.

The compounding of the first and the second component, and any third components, to be described below, is typically carried out in, e.g., an extruder, in particular a single or dual screw extruder. In the compounding process the screw extruder profile of the screw is preferably such that its dimensions will allow relatively large wood chips to move along the screw without crushing them. Thus, the channel width and flight depth are selected so that the formation of excessive local pressure increases, potentially causing crushing of the wood particles, are avoided. The temperature of the cylinder and the screw rotation speed are also selected such as to avoid decomposition of wood chip structure by excessively high pressure during extrusion. For example a suitable barrel temperature can be in the range of about 110 to 150° C. from hopper to die, while the screw rotation speed was between 25-50 rpm. These are, naturally, only indicative data and the exact settings will depend on the actual apparatus used.

A composition comprising merely the first and the second components typically is rigid. The polymer of the first component is hard.

This kind of composition is, according to the present technology, converted to a semi-rigid structure with help of at least one additional polymer or by mechanical processing, by incorporating, polymer rich regions into the material or a combination of two or more of these.

Thus, the present composite material typically comprises regions of elasticity or softness to provide for objects having properties of flexibility or semi-rigidity in at least one dimension.

Such regions of elasticity or softness can be achieved in a plurality of ways. Such regions of elasticity or softness are preferably integrated into the material, such that they are a part of the material. Thus, the composite material as such (not merely, for example, a carrier layer) exhibits said regions. The regions of elasticity or softness can be provided for example as is explained below.

In a first embodiment, the composition comprises a third component formed by a polymer different from the polymer of the first component, said polymer of the third component being capable of forming into the material regions of elasticity or softness in order to confer to the composite material mechanical properties in the range from flexibility to semi-rigidity in at least one dimension of the object at ambient temperature.

The flexible properties of the novel composition are achieved by adding an elastic or soft polymer, in the following also “third component” to the first component. The elastomer can be thermoplastic or thermosetting polymer. To maintain the general relation between polymer and reinforcing agent, a part of the first component, i.e. the low-temperature polymer, can be replaced by elastic or soft polymer, thus maintaining the volume part of the polymer in the composite material at least essentially unaltered—typically a variation of ±10% of the polymer volume is possible.

In one embodiment, three dimensional objects produced from the above embodiment exhibits unidirectionally arranged regions of elasticity or softness, preferably formed by soft polymer rich regions.

When the material is shaped into a generally planar object having a longitudinal and lateral axis, the soft (or elastic) polymer rich regions are generally unidirectional either along the longitudinal or lateral axis. The soft (or elastic) polymer rich regions can also be in form of a grid, mesh or web.

Typically, the third component is formed
by a polymer having a Shore D hardness of 27 or less, in particular 25 or less, at ambient temperature, or
by a thermoplastic elastomer.

Other examples of soft polymers include polymers exhibiting Shore A of 0 to 70 and Shore OO of 0 to 90.

The third component can be formed by a polymer selected from the group of thermoplastic polyolefin blends; polyurethanes; co-polyesters; polyamides; unsaturated or saturated rubbers, including natural rubber, silicone, and copolymers of olefins; and natural or synthetic soft material, including soft gelatin, hydrogels, hydrocolloids and modified cellulose.

The third component, i.e. the elastic or soft polymer, does not need to have melting range in same range as the first component. Typically, the third component has a melting range outside that of the first component, in particular the melting point of the polymer of the third component is higher than the melting point of the first component.

In an embodiment of a composite material according to the present technology, the third component is miscible with first component forming a homogenous matrix when processed at elevated temperatures.

In another embodiment, the third component is immiscible with the first component forming phase-separated zones or regions within the first component.

Based on the above, in one embodiment of the present technology, the composite material comprises about
10 to 70 parts by weight of a biodegradable polyester;
25 to 60 parts by weight of wood particles; and
5 to 40 parts by weight of a soft or elastic polymer.

Preferably the soft or elastic polymer together with the biodegradable polyester make up a majority of the composition (i.e. more than 50% by weight of the total weight of the composition). In a particular preferred embodiment, the soft or elastic polymer together with the biodegradable polyester make up at least 53% and up to 70%, for example 55 to 70%, by weight of the total weight of the composition. The soft or elastic polymer generally forms 5 to 50%, in particular 10 to 40%, for example 15 to 30%, by weight of the total weight of the biodegradable polyester together with the soft or elastic polymer.

It is possible to incorporate further polymers into the composition. In one embodiment, the composition comprises 3 to 30 parts by weight, of a fourth component comprising a thermoplastic polymer different from that of the first and the third component. Such a component can be used for achieving improved mechanical properties of the matrix polymer. It is also possible to use a fourth polymer to modify the surface properties (for example properties of adhesion) of the composition.

Another method to achieve a semi-rigid composite according to invention is to provide punctuation of the composite, for example with unidirectional incisions. The size, frequency and perforation pattern may vary. In a more preferred embodiment of invention, both elastic or soft polymer as third component and punctuation are used to enhance the overall flexibility of a orthopedic support according to invention.

The incisions may be manufactured into the composite profile with an incision device, examples of suitable equipment include a rolling cylinder or a press equipped with blades, water jet, and laser cutting.

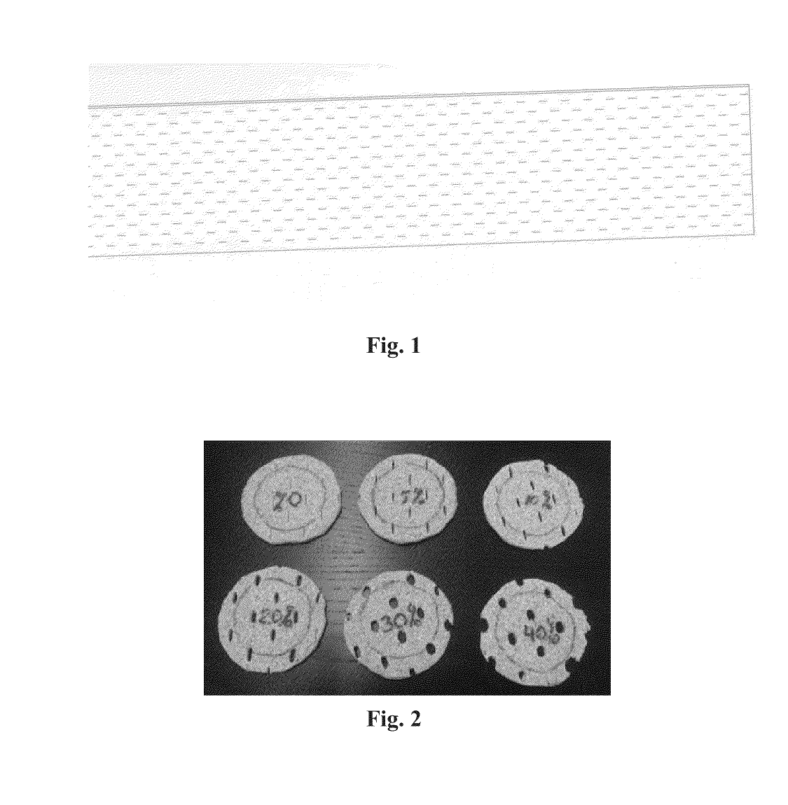

Thus, in one embodiment, the composite material comprises perforations, for example in the form of incisions, in particular unidirectional incisions, forming a region of flexibility. An example of such incisions is shown in the attached drawing (FIG. 1).

Typically, the incisions have a width of 0.1 to 1 mm, preferably 0.3 to 0.8 mm, and a length of 4 to 20 mm.

The incisions can be made with a blade, the surface area of which incisions being on the blade ingoing side about 1 to 10 mm², preferably 2.5 to 8 mm².

The composite material according to any of the preceding claims, comprising incisions, the amount of the incisions/10 cm² being generally 20 to 100, preferably 30 to 70.

Stretching the material at application temperature, the practician or clinician may adjust the flexibility of a set product by controlling the aperture openings.

The particular advantage of incorporating incisions into the material is that upon stretching when the material is applied on a patient, the incisions will yield openings which give the material properties of breathability. Thereby skin maceration can be avoided.

The shape of the openings or apertures formed by stretching of the incision can be, for example, round, rectangular, square, diamond, hexagonal, oval, slot or ornamental perforation. The surface area of one hole should be generally about 3 to 30 mm² and amount of the holes is kept between 20 holes/10 cm² and 100 holes/10 cm². The total open area is less than 10 percentage of the whole surface area.

Openings can also be formed directly to different shapes for example, round, rectangular, square, diamond, hexagonal, oval, slot or ornamental perforation (without incisions).

FIG. 4 shows the effect of latitudinal stretching on a longitudinally incised composite material.

Manufacturing of an adhesive composite material comprising of only adhesive thermoplastic polymer and wood component by extrusion is a straightforward process.

When adhesive polymer is partially replaced with any non-adhesive polymer e.g. elastomers properties of adherence of the composite are compromised. When the composite material contains 15% by weight or more of the non-adhesive elastomer adhesion is significantly reduced.

In an advantageous embodiment, a composite material which has properties of softness, contains about 17.5 to 30 w-% of a soft polymer component. The adhering feature can be restored by coating the surface of the composite material with an adhesive thermoplastic polymer e.g. polycaprolactone. The surface of the composite material can be coated completely or partially as stripes.

The coating is required on both sides of the composite blank to be feasible for immobilization applications. The easiest way to manufacture such a profile is by using extrusion process, in where adhering coating component can be added onto the profile by using appropriate die unit. The coating component can be fed to the die by using separate extruder. The coating component can comprise only of one adhering homopolymer, e.g. polycaprolactone, or it can be a blend of several polymers, adhesive and non-adhesive, as well. The profile is preferable only partially coated as stripes in such manner that coatings on the top and bottom surfaces are not in contact with each other.

Thus, a composite material according to the present technology may comprise superficial adhesion stripes enhancing properties of self-adherence of the material.

Since the possibility to combine the semi-rigid compositions with rigid compositions is advantageous for the application, the reduced tackyness or self-adhesion at application temperatures, the restoration of adhering properties may be recommended.

To lower the risk of tearing of the composite blank during applying at ˜65° C. composite material may contain additional thermoplastic polymer having melting point above 100° C. and tensile strength more than 20 N/mm² (ISO 527).

The following non-limiting examples illustrate the invention:

### EXAMPLE 1

A number of composite materials were produced having the following composition:

30 to 40 w-% wood particles;

5 to 10 w-% thermoplastic polymer having a melting point above 100° C.;

15 to 30 w-% of a thermoplastic elastomer; and

30 to 50 w-% thermoplastic polymer (polycaprolactone) having a melting point of ˜60° C.

In one specific embodiment, the composition contain about 35 to 40 wt-% poly-caprolactone, 30 to 35 wt-% wood chips, 5 to 7.5 wt-% aliphatic-aromatic copolyester and 20 to 25 wt-%) polyolefin elastomer.

One of the compositions was compounded to composite blank with dimension 150×800 mm and thickness approximately 4 mm. This composition comprised 37.5 wt-% of high Mw polycaprolactone, 33 wt-% platy woodchips, 23% polyolefin elastomer and 6.5% aliphatic-aromatic co-polyester as a fourth component.

The blank was exhibiting desired semi-rigid mechanical properties comparable with synthetic semi-rigid casting materials when set. The blank was further prepared to specimens for mechanical tests (Example 2).

### EXAMPLE 2

For determining the rigidity, so called ring stiffness specimens were prepared from a composition manufactured in Example 1. A test protocol according to ISO 9677:2007standard was followed. Series of cylindrical test specimen with a diameter of app. 75 mm and a length of 80 mm were manufactured according to instructions from manufacturers. A synthetic casting tape, Softcast™ (manufactured by 3M) was selected as a control sample for commercially available semi-rigid casting tapes. A biodegradable wood-plastic cast, WOODCAST® 2 mm (manufactured by Onbone Oy) was selected as a control sample for rigid casts. Sample cylinder wall thickness was adjusted to 2.0 mm with each specimen.

With Softcast™ four layers of laminated casting tape corresponds with wall thickness of 2 mm.

The semi-rigid composite blank was tested both as intact and incised. Two lengths of incisions, namely 5 mm and 10 mm were punctuated with a blade tool to composite blank. Interval of 5 mm incisions were 15 mm, adjacent rows of incisions were shifted by 5 mm. Interval of 10 mm incisions were 30 mm, adjacent rows of incisions were shifted by 10 mm.

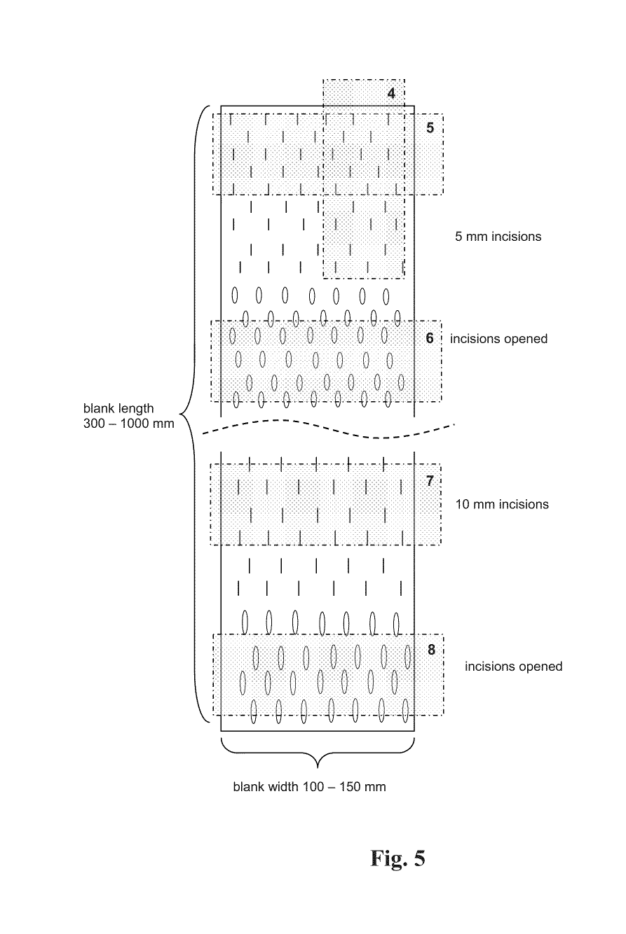

The stiffness tests of incised blanks were tested in directions parallel and perpendicular to incisions. Moreover, specimens with stretch-opened incisions were prepared in order to demonstrate the influence of aperture when opened to approximately 5% aperture area over the total area of a specimen. The parallel test specimens correspond clinically with bending forces along an arm or limb. Perpendicular tests correspond clinically with circumferential bending forces around a limb. The specimen preparation patterns are illustrated in FIG. 5.

Ring stiffness was measured by recording the force and the deflection while compressing the cylinder at a constant deflection speed at vertical direction. A cross head speed of 20 mm/min was used in this test and the deflection was carried on until 50% deflection of the diameter of the cylinder was achieved. A plot of force versus deflection was generated using materials testing machine (LLOYD LR30K, Lloyd instruments, Southampton, UK) with 1 kN load cell for each specimen. In each series six samples were tested. The ring stiffness was calculated as a function of the force necessary to produce a 3% diametric deflection to the ring.

As it is seen in results, Table 1, the semi-rigid composition exhibits the reduction of stiffness by approximately 50% as compared to rigid sample of same thickness. In the incised specimens the stiffness is further reduced to the same magnitude with semi-rigid synthetic casting tape.

TABLE 1
Comparison of semi-rigid compositions with a rigid composition

| Sample | | Openings | Stretch | aperture | Ring Stiffness |
|---|---|---|---|---|---|
| # | Description | (YES/NO) | open | (% of area) | (kN/m² w/STD) |
| 1 | Softcast ™ (3M) semi-rigid casting tape | NO | n.a. | n.a. | 2.5 ± 0.2 |
| 2 | WOODCAST ® 2 mm (by Onbone) | NO | n.a. | n.a. | 33.0 ± 1.2 |
| 3 | Semi-rigid composite | NO | n.a. | n.a. | 16.4 ± 2.2 |
| 4 | Semi-rigid with 5 mm parallel incisions | YES | NO | 1% | 8.6 ± 1.0 |
| 5 | Semi-rigid composition with 5 mm | YES | NO | 1% | 3.0 ± 0.3 |
| 6 | perpendicular incisions | | YES | 5% | 2.9 ± 0.7 |
| 7 | Semi-rigid composition with 10 mm | YES | NO | 1% | 3.6 ± 0.4 |
| 8 | long perpendicular incisions | | YES | 5% | 2.6 ± 0.1 |

### EXAMPLE 3

Incised composite samples were either used as native, or stretched to enable opening of the voids in structure. The stretching ratios of the samples were as follows; 0%, 5%, 10%, 20%, 30%, and 40%. In FIG. 2 the actual test specimens are shown.

The total area of the voids in samples was first measured by first copying the samples in copying machine. From the copied papers the 2D-pictures of samples the weight of whole sample areas was first measured. The void areas were then cut out from the paper copies and the weights of these samples (with void areas removed) were then measured. This test was repeated three times with each of the samples, and the average void area was then calculated for each sample type.

To perform the vapour permeability analysis a thermo gravimetric analysed, HR73 (Mettler Toledo, USA) was used. The constant temperature of 50° C. was used with all the samples through the tests. From each sample type a circular sample with diameter approximately 60 mm was cut. 2 ml of distilled water was laced to a petri dish and the sample was placed over the dish. The sample was further sealed with aluminium tape from the sides and the edges of sample. With each sample care was taken that the open non-sealed area was constant to enable accurate comparative results. The petri dish was then placed into analyzer and the weight change was recorded over 60 minutes with 5 minutes time intervals. From the obtained data the vapour permeation rate was then calculated.

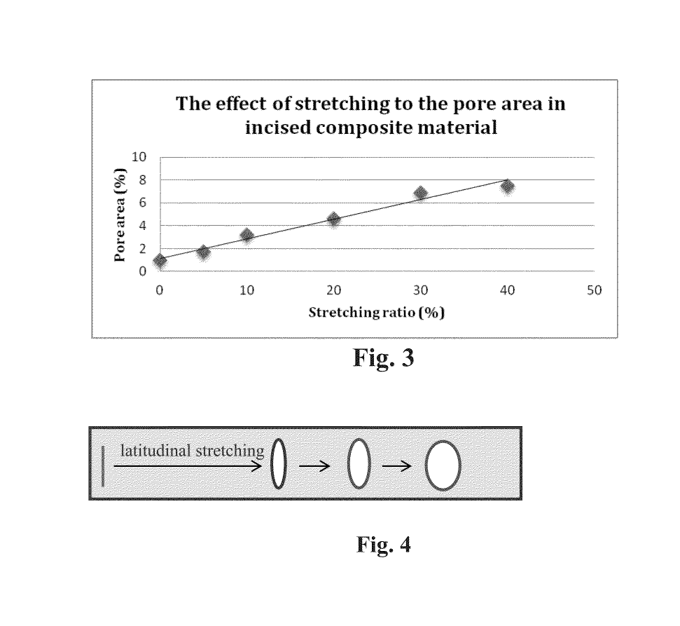

The effect of stretching to the pore area of incised composite samples is seen in FIG. 3. As will appear from the graph the stretching ratio linearly affects the pore area of samples.

In non-stretched samples pore area is approximately 1% and in samples with 40% of stretching the pore area is approximately 8%.

### EXAMPLE 4

Utilisation of Semi-Rigid Composition in Orthopedic Casting

Steps:

1. A manufactured semi-rigid composite blank (with black stripes) is heated up to its application temperature.

2. A warm semi-rigid composite blank is combined with a pre-shaped rigid wood-plastic composite Woodcast® 2 mm.

3. The warm materials are applied on patients leg padded appropriately.

4. An ankle boot cast with rigid and semi-rigid regions is finally equipped with appropriate bandaging and Velcro tapes.

### INDUSTRIAL APPLICABILITY

The present materials can be used in splints and circumferential casts. In sport appliances, such as grips for rackets in rackets sports, as well as in the above-mentioned foot-supporting applications, the capability of the material easily to be formed and exhibiting a degree of softness or elasticity is of particular use.

### CITATION LIST

### Patent Literature

WO 2006/027763A2,

WO 2007/035875,

US 2008/0262400,

US 2012/0071590

WO 94/03211

WO 2012/032226

1. A composite material in the shape of a three-dimensional object, comprising a first component formed by a polymer and a second component formed by a reinforcing material, wherein
said first component comprises a thermoplastic polymer selected from the group of biodegradable polyesters and mixtures thereof, and
said second component comprises particles of a woody material, having a smallest dimension greater than 0.1 mm,
said composite material further comprising
regions of elasticity or softness to provide for objects having properties of flexibility or semi-rigidity in at least one dimension, and
said material being moldable and workable at temperatures below 70° C.

2. The composite material according to claim 1, further comprising a third component formed by a polymer different from the polymer of the first component, said polymer of the third component being capable of forming into the material regions of elasticity or softness in order to confer to the composite material mechanical properties in the range from flexibility to semi-rigidity in at least one dimension of the object at ambient temperature.

3. The composite material according to claim 1, wherein the biodegradable polyester has a melting point below 70° C. and higher or equal to 55° C.

4. The composite material according to claim 1, wherein biodegradable polyester forms the matrix of the composite material.

5. The composite material according to claim 1, wherein the first component forms the matrix of the composite material, and the second component exhibits a microstructure which is discontinuous.

6. The composite material according to claim 1, wherein the biodegradable polyester is selected from the group of epsilon-caprolactone homopolymers, blends of epsilon-caprolactone homopolymers and other biodegradable thermoplastic homopolymers, with 5-99 wt % of an epsilon-caprolactone homopolymer and 1-95 wt % of a biodegradable thermoplastic polymer, and copolymers of epsilon-caprolactone homopolymer and any thermoplastic biodegradable polymer, with 5 to 99 wt % of repeating units derived from epsilon-caprolactone and 1 to 95 wt % repeating units derived from other polymerizable material.

7. The composite material according to claim 1, further comprising a first polymer component having a melt flow index of 0.3-2.3 g/min (at 80° C.; 2.16 kg).

8. The composite material according to claim 1, wherein the third component is formed by a soft or elastic polymer.

9. The composite material according to claim 1, wherein the third component is formed
by a polymer having a Shore D hardness of 27 or less at ambient temperature, or
by a thermoplastic elastomer.

10. The composite material according to claim 1, wherein the third component is formed by a polymer selected from the group of thermoplastic polyolefin blends; polyurethanes; co-polyesters; polyamides; unsaturated or saturated rubbers, including natural rubber, silicone, and copolymers of olefins; and natural or synthetic soft material, including soft gelatin, hydrogels, hydrocolloids and modified cellulose.

11. The composite material according to claim 1, wherein the third component has a melting range outside that of the first component.

12. The composite material according to claim 1, wherein
the third component is miscible with first component forming a homogenous matrix when processed at elevated temperatures; or
the third component is immiscible with the first component forming phase-separated zones or regions within the first component.

13. The composite material according to claim 1, further comprising
10 to 70 parts by weight of a biodegradable polyester;
25 to 60 parts by weight of wood particles; and
5 to 40 parts by weight of a soft or elastic polymer.

14. The composite material according to claim 1, wherein the three dimensional object exhibits unidirectionally arranged regions of elasticity or softness, preferably formed by soft polymer rich regions.

15. The composite material according to claim 1, wherein the material is shaped into a generally planar object having a longitudinal and lateral axis and wherein the soft polymer rich regions are unidirectional either along the longitudinal or lateral axis.

16. The composite material according to claim 1, wherein the material is shaped into a generally planar object having a longitudinal and lateral axis and wherein the soft polymer rich regions are in form of a grid, mesh or web.

17. The composite material according to claim 1, wherein the woody material comprises platy wood particles or particles obtained from such wood particles, by crushing, said particles forming 30 to 100% of the total weight of the second component.

18. The composite material according to claim 1, further comprising 10 to 50 parts by weight, of a fourth component comprising a thermoplastic polymer different from that of the first and the third component.

19. The composite material according to claim 1, further comprising superficial adhesion stripes enhancing properties of self-adherence of the material.

20. The composite material according to claim 1, wherein the composite material is shaped into a planar blank exhibiting polymer rich longitudinal regions for improving properties of anatomical shape ability (contouring).

21. The composite material according to claim 1, further comprising perforations forming a region of flexibility.

22. The composite material according to claim 1, further comprising incisions having a width of 0.1 to 1 mm and a length of 4 to 20 mm.

23. The composite material according to claim 1, further comprising incisions made with a blade, the surface area of which incisions being on the blade ingoing side being 1 to 10 mm².

24. The composite material according to claim 1, further comprising incisions, the amount of the incisions/10 cm² being 20 to 100.

25. The composite material according to claim 1, wherein the individual wood particles have at least two dimensions greater than 1 mm and one greater than 0.1 mm.

26. The composite material according to claim 1, wherein the wood particles are capable of being orientated and aligned in a melt flow of the thermoplastic polymer.

27. The composite material according to claim 1, wherein the wood particles comprise chips of hardwood, softwood or a combination thereof.

28. The composite material according to claim 1, further comprising a particulate material, a fibrous material or a combination thereof as a reinforcing component, said component forming 1 to 15% of the weight of the second component.

29. The composite material according to claim 1, said material being formable at a temperature of 50 to 70° C. and being rigid at a temperature of less than 50° C.

30. The composite material according to claim 1, wherein a majority of wood particles is greater in size than powder, being granular or platy and making up more than 70% of the woody material, said woody material making up more than 70%) of the second component.

31. A method of producing a composite material in the shape of a three-dimensional object comprising combining
a first component which is formed by a thermoplastic polymer selected from the group of biodegradable polyesters and mixtures thereof with
a second component which is formed by particles of a woody material, having a smallest dimension greater than 0.1 mm to produce a composite material,
shaping the material into a three dimensional object, and
introducing into the composite material regions of elasticity or softness to provide the objects with properties of flexibility or semi-rigidity in at least one dimension,
wherein the composite material comprises a first component formed by a polymer and a second component formed by a reinforcing material, wherein
said first component comprises a thermoplastic polymer selected from the group of biodegradable polyesters and mixtures thereof, and
said second component comprises particles of a woody material, having a smallest dimension greater than 0.1 mm,
said composite material further comprising
regions of elasticity of softness to provide for objects having properties of flexibility or semi-rigidity in at least one dimension, and
said material being moldable and workable at temperatures below 70° C.

32. The method according to claim 31, wherein the step of introducing into the composite material regions of elasticity or softness comprises incorporating into the composite materials a third component a third component formed by an elastic or soft thermoplastic polymer.

33. The method according to claim 32, wherein the polymer of the third component is homogeneously distributed within the polymer of the first component, or the polymer provides regions rich within said polymer of said first component.

34. The method according to any of the claims 31, wherein the biodegradable polyester forms the matrix of the composite material.

35. The method according to claim 31, wherein the first component forms the matrix of the composite material, and the second component exhibits a microstructure which is discontinuous within the matrix.

36. The method according to claim 31, wherein perforations are formed into the composite material.

37. The method according to claim 36, wherein the incisions are made with a blade, said incisions having a width of 0.1 to 1 mm and a length of 4 to 10 mm.

38. The method according to claim 36, wherein the size of the individual incisions are different on the opposite sides of the composite profile.

39. Splints and circumferential casts, comprising a composite material in the shape of a three-dimensional object, the composite material comprising a first component formed by a polymer and a second component formed by a reinforcing material, wherein
said first component comprises a thermoplastic polymer selected from the group of biodegradable polyesters and mixtures thereof, and
said second component comprises particles of a woody material, having a smallest dimension greater than 0.1 mm,
said composite material further comprising
regions of elasticity or softness to provide for objects having properties of flexibility or semi-rigidity in at least one dimension, and
said material being moldable and workable at temperatures below 70° C.
